(19) United States
(12) Patent Application Publication
(10) Pub. No.: US 2010/0229858 A1
(43) Pub. Date: Sep. 16, 2010

(54) AQUATIC HEADGEAR

(76) Inventor: TROY L. WHEELWRIGHT, Amesbury, MA (US)

Correspondence Address: BOURQUE & ASSOCIATES;INTELLECTUAL PROPERTY ATTORNEYS, P.A., 835 HANOVER STREET, SUITE 301, MANCHESTER, NH, 03104, US

(21) Appl. No.: 12/603,228

(22) Filed: Oct. 21, 2009

Related U.S. Application Data
(63) Continuation-in-part of application No. 11/387,141, filed on Mar. 22, 2006, now abandoned.
(60) Provisional application No. 61/107,120, filed on Oct. 21, 2008.
(60) Provisional application No. 61/111,404, filed on Nov. 5, 2008.

(51) Int. Cl. B63C 11/16; A61F 9/02

(52) U.S. Cl. 128/201.11; 2/426

(57) ABSTRACT

An improved aquatic device for use with goggles features a support base sized and shaped to fit near the back of a user's head and is secured against the user's head by way of straps. Various equipment may be removably secured to the support base including a snorkel and/or a light by means of a universal interconnection mechanism. The universal connection system allows the various equipment to be removably secured to the support base such that the user to select the equipment most suited to the intended application. One portion of the universal connection system is found on the support base while the other is located on the equipment such as a snorkel or light. The snorkel preferably includes a dry top that prevents water from entering the air passageway. The dry top includes a float mechanism and an inlet closure mechanism both pivoting from a common axis. The dry top may also include a one-way rubber valve that allows air to only enter the dry top and not exhaust. Flexible tubes connect the snorkel secured to the support base to the mouthpiece. In one embodiment there are two tubes; one for air inlet and one for air exhaust. The flexible tubes are arranged such that they extend behind the user's head and beneath the user's jaw. As a result, drag is significantly reduced and mobility and comfort are increased.

### CROSS-REFERENCE TO RELATED APPLICATIONS

This application is a continuation-in-part of U.S. patent application Ser. No. 11/387,141 entitled “AQUATIC HEADGEAR” filed on Mar. 22, 2006, and claims the benefit of U.S. Provisional Application Ser. No. 60/748,711 entitled “HEAD MOUNTED FLEXIBLE SNORKLE FOR SWIMMERS AND DIVERS” filed on Dec. 9, 2005; U.S. Provisional Application 61/107,120 entitled “AQUATIC HEADGEAR” filed Oct. 21, 2008; and U.S. Provisional Application 61/111,404 entitled “MASK STRAP ASSEMBLY′ filed Nov. 5, 2008.

### TECHNICAL FIELD

The present invention relates to snorkeling and scuba diving equipment and more particularly, relates to a head mounted aquatic snorkel system.

### BACKGROUND INFORMATION

It can be appreciated that swimming snorkels have been in use for years. Traditionally, swimming snorkels are comprised of a semi-hard rubber or plastic tube that is used by divers or snorkelers for breathing while swimming face down in the water. The existing products usually have a molded rubber portion attached to the lower end of the snorkel tube that is angular in shape for directing the mouthpiece to the divers mouth. The snorkel tube is usually secured to the goggle strap on one side of the head by a rubber connector or clip.

Some devices found in the market place have improvement features that include a purge valve located next to the mouthpiece for purging excess water from the breathing tube. Also, some units incorporate a valve mechanism on the top of the snorkel which helps prevent water from entering while breathing. Some existing units have a snorkel tube that is shaped so that they more easily form to the side of the divers head.

While these traditional snorkels are generally effective, they suffer from numerous limitations and disadvantages. For example, conventional swimming snorkels are generally awkward and uncomfortable to use and difficult to adjust. Water flowing past the swimmer's head exerts a lot of force against the snorkel. Because the snorkel is secured to the goggle strap at a single point on the goggle strap, much of this force is transmitted by the snorkel to the goggle strap thereby causing discomfort for the user. Additionally, the proximity of the snorkel to the goggles can make fitting and sealing the goggles to the user's head difficult and can limit the mobility of the user. Moreover, when the snorkel is not in use, the hard tube hangs like a weight on the side of the divers goggles causing further discomfort and flops around thereby getting in the way. As a result, even though most scuba/snorkeling organizations suggest wearing a snorkel while scuba diving, many scuba divers remove their snorkels.

Another limitation of the known snorkeling incorporating a device to keep the snorkel breathing tube “dry” when the swimmer is submerged under water is that these devices only work when the swimmer is a face down position. If the swimmer rolled over onto his or her back or side, the traditional float mechanism which keeps the snorkel inlet dry will open up allowing water to enter the snorkel and which must be purged before breathing through the snorkel can resume.

Yet another limitation of the known snorkels is that it is often difficult to identify one individual from another. For safety reasons, divers and snorkelers generally team up with at least one swim “buddy”. It is the responsibility of each swim buddy to watch over the other and come to his/her aid. Swimmers commonly wear wet suits (often having hoods which cover the user's head) to protect against the cold and these wet suits often look very similar. As a result, identifying a person's swim buddy from the others while underwater is often very difficult and can lead to a very dangerous situation if the swim buddies become separated.

A further limitation of the known snorkel and diving systems is that they are difficult to use at night. When diving at night, it is necessary to have a source of light to illuminate the diver's surroundings. As anyone who has dove can attest, divers are generally required to carry numerous other pieces of equipment, much of which must be held in order to use. The known light sources for diving are typically waterproof flashlights. The user must generally hold the flashlight at all times, thereby limiting the diver's ability to utilize other pieces of equipment.

Lastly, it has been found that snorkel systems which utilize a single tube for both inhaling and exhaling are inefficient and cause the user to inhale previously exhaled air which is lacking in sufficient oxygen for the swimmer.

Accordingly, there exists a need for an improved aquatic device for swimmers and divers that will overcome the shortcomings of the prior art devices described above. The improved aquatic device should preferably eliminate the need to secure the snorkel to the side of the strap of the goggle on the side of the user's head. Additionally, the improved aquatic device should preferably position the snorkel on the back of the head instead of on the side of the face, thereby making the snorkel more comfortable and freeing up space around the mask and face area making it easier to adjust the mask for a leak free fit. The improved aquatic device should also preferably eliminate the hard plastic or rubber snorkel tube that extends up the side of the face as used in known designs thereby increasing the user's comfort. An improved aquatic device should also include a mechanism to ensure that when the user is submerged under water, the inlet will stay dry. Lastly, an aquatic device with the improved features required also include separate inlet and exhaled pathways and appropriate valving to insure that air always enters one passageway and is exhaled through another.

There also exists a need for an improved aquatic device that eliminates the rigid tubing used in the known snorkels, hereby further increasing the user's comfort and mobility. The improved snorkel system should preferably prevent or reduce the tendency for the snorkel to pull on the goggle strap and cause discomfort. There also exists a need for an improved snorkel system that can be permanently secured to the back of a divers hood for use by scuba divers in cold water environments.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by any allowed patent claims and their legal equivalents.

### SUMMARY

According to one object of the invention described herein, the invention is designed to provide an ultra secure surface on the posterior and superior portion of a swimmer or divers head for the primary purpose of attaching head mounted snorkels, lights, cameras, and other such equipment.

Another object of the invention is to provide a secure, comfortable, and easy adjusting strap for masks and goggles.

According to one embodiment, the present invention features an aquatic device comprising a support base sized and shaped to substantially fit only over a posterior superior portion of a user's head and means for connecting the support base to a goggle strap. The support base may be generally flexible or generally rigid, and optionally includes a layer of a non-slip material (such as neoprene) along the interior surface.

The goggle strap may include a first and a second separate strap permanently secured to a first and a second generally opposite side of the support base, respectively. Alternatively, at least one channel may be disposed within the support base that is sized and shaped to accept at least a portion of the goggle strap such that the goggle strap passes through at least one a portion of the support base.

The aquatic device may also feature a snorkel secured to and extending generally outwardly and away from the support base and at least one flexible tube. The flexible tube has a first end connected to an air passage of the snorkel and a second end connected to a mouthpiece. The air passageway of snorkel optionally includes a first aperture disposed proximate a distal end of the snorkel and at least a second aperture fluidly connected on opposite ends of the air passageway, wherein the at least a second aperture is disposed proximate the support base. The second aperture is preferably disposed proximate a back region of the support base such that when the aquatic device is worn on the user's head, the second aperture generally faces towards a neck of the user's head. The flexible tube is preferably sized and shaped such that when the aquatic device is worn on the user's head, the flexible tube is substantially disposed behind the user's head, around a chin of the user, and connects with the mouth piece. The aquatic device may feature a first and a second flexible tube adapted to be disposed on a first and a second generally opposite side of the user's head when worn.

The support base may optionally include means for removably securing the snorkel to the support base. Any means to accomplish this is considered within the scope of the present invention. For example, for exemplary purposes only, the support base may be removably secured to snorkel by way of a channel (preferably disposed within the support base) and a pin sized and shaped to fit within the channel. Optionally, a biased tap is disposed proximate an open end of the at least one channel and urges the snorkel within the channel.

The snorkel may also include a splashguard or dry top. The splashguard preferably includes a flanged region disposed proximate a distal of the air passageway of the snorkel, a closure flap and a buoyant float moveably disposed about an exterior portion of the snorkel. The buoyant float and closure flap are tentatively connected to the top of the snorkel at the same pivot point or axis of rotation. When the buoyant float is above water, gravity moves the buoyant float and closure flap to a first position such that the air passageway is substantially unobstructed. When the buoyant float is below water, gravity moves the buoyant float to a second position such that the buoyant float pushes the closure flap and engages the closure flap against the flanged region of the snorkel, thus sealing the air passageway. If the swimmer turns onto his or her back, although the buoyant float may fall away from its position against the closure flap, the pressure of the water will keep the closure flap closed thus sealing the air passageway until the end of the snorkel exits the water.

The aquatic device may optionally includes at least one light. A power source may be located proximate the support base or remotely from the support base. The light is preferably removably connected to the support base, though it may also be permanently secured to the support base.

According to another embodiment, the present invention features a sport apparatus. The sport apparatus includes a goggle, a strap secured to the goggle, a support base secured to the strap and sized and shaped to substantially fit only over a posterior superior portion of a user's head, and engagement means for removably securing an object to the support base. The object preferably includes and snorkel and/or a light.

According to another aspect of the present invention, the aquatic device may be provided with a separate air inlet path by which the user draws air into his or her lungs, and a separate air exhaust or exhale path which allows spent air from the user to immediately exit the aquatic device by means of a separate path, thereby not placing exhausted, oxygen depleted air into the air intake for the user to have to “re-breath”.

### BRIEF DESCRIPTION OF THE DRAWINGS

These and other features and advantages of the present invention will be better understood by reading the following detailed description, taken together with the drawings wherein:

FIG. 1 is a side plan view of one embodiment of the improved aquatic device according the present invention;

FIG. 2 is a top plan view of one embodiment of the support base of the improved aquatic device according to the present invention;

FIG. 3 is a top plan view of another embodiment of the support base of the improved aquatic device according to the present invention;

FIG. 4 is a top, front plan view of one embodiment of the support base of the improved aquatic device in conjunction with a snorkel according to the present invention;

FIG. 5 is a top, back plan view of one embodiment of the support base of the improved aquatic device in conjunction with a snorkel having a splash guard according to the present invention;

FIG. 6 is a perspective rear view of one embodiment of the mouthpiece of the improved aquatic device according to the present invention;

FIG. 7 is a top plan view of one embodiment of the support base of the improved aquatic device having a connection system according to the present invention;

FIG. 8 is a front plan view of the support base of the improved aquatic device having a connection system shown in FIG. 7 according to the present invention;

FIG. 9 is a top, front plan view of one embodiment of the support base of the improved aquatic device and a removable snorkel according to the present invention;

FIG. 10 is a front plan view of one embodiment of the removable snorkel of the improved aquatic device according to the present invention;

FIG. 11 is a front view of one embodiment of the removable light of the improved aquatic device according to the present invention;

FIG. 12 is a side perspective view of a dry top for a snorkel in accordance with one aspect of the present invention;

FIG. 13 is a side perspective review of the dry top of FIG. 12 with the enclosure cover removed and the float and closure flap in the closed position;

FIG. 14 is a side perspective view of the dry top of FIG. 13 with the float in the open position and the closure flap in the closed position;

FIG. 15 is a side perspective view of the dry top of FIG. 14 with the float and closure flap in the open position;

FIG. 16 is a cross-sectional view of the dry top of FIG. 13 showing the exhale prevention flap located in the interior of the dry top and the inhale tube closure flap and float in the closed position, blocking the air inlet;

FIG. 17 is a perspective view of the aquatic device in accordance with the present invention showing an exhale tube and valve path;

FIG. 18 is a front perspective view of a portion of the aquatic device showing the exhale flap located on the exhale tube path;

FIG. 19 is a side perspective view of the aquatic headgear according to yet another embodiment of the present invention incorporating a design or character element;

FIG. 20 is a side view of the easy adjusting mask strap according to one feature of the present invention;

FIG. 21 is a side view of the easy adjusting mask strap according to another feature of the present invention including a breathing tube and an accessory mounting bracket; and

FIG. 22 is a side view of the easy adjusting mask strap according to one feature of the present invention mounted to a diver's hood piece.

### DETAILED DESCRIPTION OF THE PREFERRED EMBODIMENTS

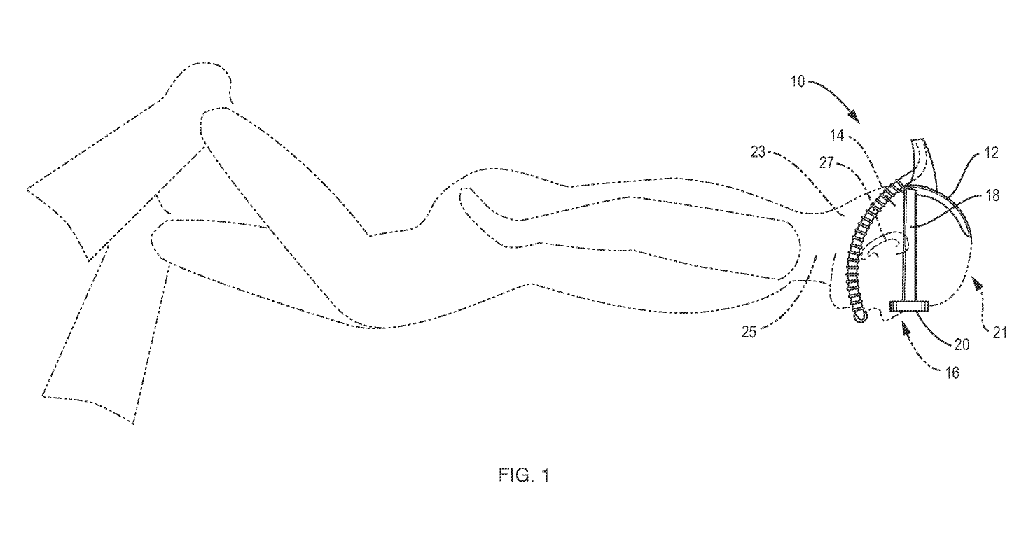

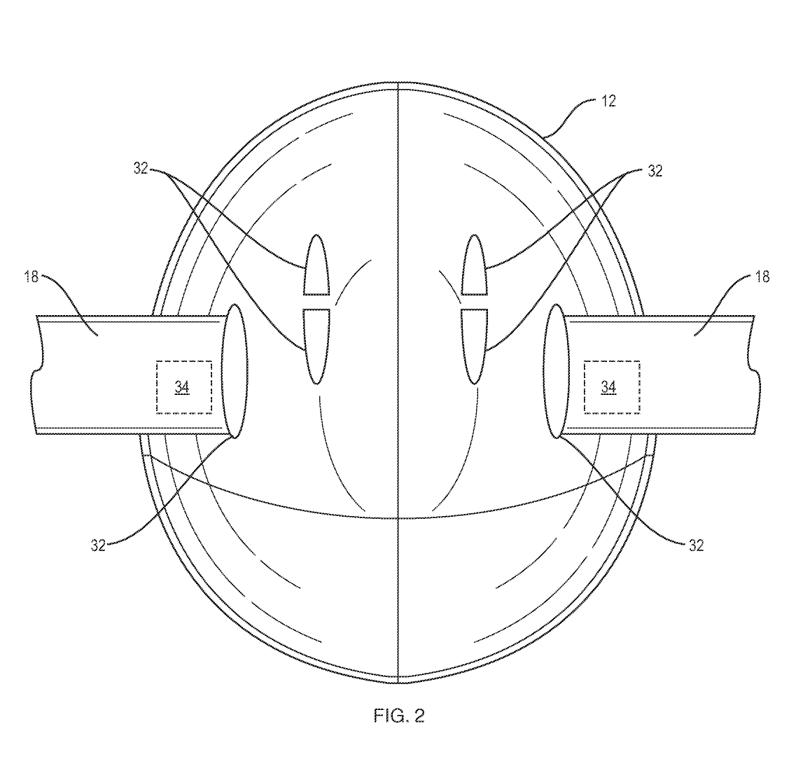

According to one embodiment, the present invention features an improved aquatic device 10, FIG. 1, for use with snorkeling or scuba diving that features a support base 12 adapted to disposed proximate the back region 14 of a user's head 16 which is held in place by way of a strap 1B of a pair of goggles 20. Referring specifically to FIGS. 2-7, the support base 12 has a shape contoured to fit over at least a portion of the back 14 (FIG. 1) of a user's head 16 and preferably conforms to the user's head 16. As will be described in greater detail hereinafter, locating the support base 12 proximate the back of the user's head 16 facilitates the use of a snorkel 22 (FIG. 1) and/or other devices, especially when the user's head 16 is face down in the water.

In the exemplary embodiment, the support base 12 has a generally dome-like shape that extends over the posterior superior portion of the user's head 16 such that the support base cradles the user's head 16. The support base 12 is not intended to serve as a protective gear, but rather it is important to note that the support base 12 should be sized and shaped to facilitate movement and/or range of motion of the user's head 16. The support base 12 will preferably cradle the user's head, from a top region 21 (FIG. 1) near the back of a user's head 16 to the approximate joint 23 between the user's head 16 and neck 25, but not over the users ears 27. The support base 12 is preferably large such that it substantially does not move relative to the user's head 16 during use, though it may be enlarged to create a pleasing aesthetic appearance. The support base 12 may, however, be incorporated into a diving hood, however, the placement of the support base 12 is preferably disposed proximate posterior superior portion of the user's head 16.

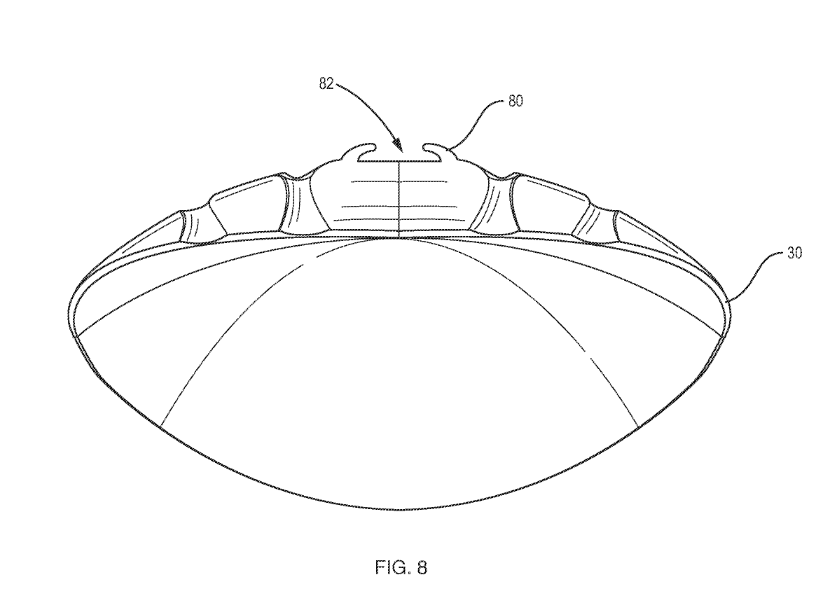

In the preferred embodiment, the support base 12 may be constructed from a flexible, resilient material such as, but not limited to, plastic, neoprene, rubber, or any other suitable material. Alternatively, the support base 12 may be constructed from a generally rigid material. Optionally, a lower or interior surface 30 (best seen in FIG. 8) may include a non-slip, high-friction and/or cushioning surface such as, but not limited to, silicon rubber or neoprene. The non-slip and/or cushioning surface further enhances the user's comfort and reduces the likelihood of the support base 12 moving relative to the user's head 16.

As discussed above, the support base 12 is held in place by means of the straps 18 of the goggles 20. According to one embodiment, the strap 18, FIG. 2, may be secured to the support base 12 by means of one or more apertures, channels, or slots 32 sized and shaped to accept at least a portion of the strap 18. The apertures 32 may be located in various regions of the support base 12 to allow the strap 18 to be secured to various areas of the support base 12. This also allows the location of the support base 12 with respect to the user's head to be slightly altered to better fit the contours of the user's head 16, thereby increasing the user's comfort.

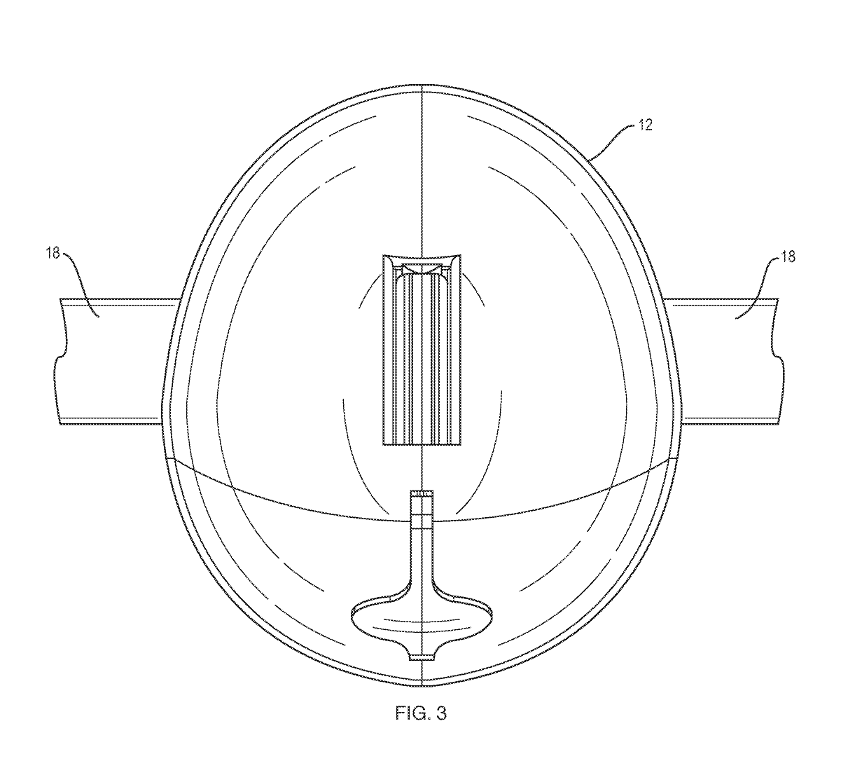

The strap 18 may also be secured to the support base 12 by other methods. For example, the strap 18 and the base 12 may feature one or more hook and loop type fasteners, buttons, or the like 34. Alternatively, the strap 18, FIG. 3, may be secured substantially permanently to the support base 12 using an adhesive, welding, mechanical clamp, or the like.

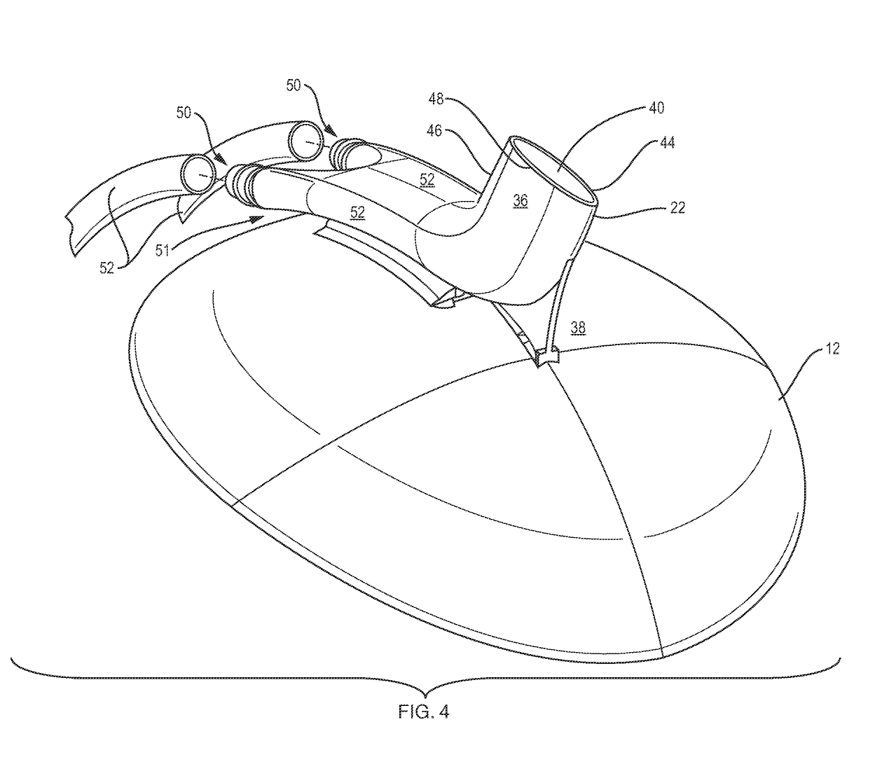

According to one embodiment, the improved aquatic device 10, FIG. 1, optionally includes a snorkel 22. In a first embodiment, the snorkel 22, FIG. 4, may be permanently secured to (or an integral unit with) the support base 12. The snorkel 22 preferably includes an elongated region 36 extending generally upwards and away from a top surface 38 of the support base 12. The exact angle that the snorkel 22 extends outwardly from the support base 12 will depend on the intended location of the support base 12 and the snorkel 22 as well as the overall dimensions of the snorkel 22, and is within the knowledge of one of ordinary skill in the art. For illustrative purposes only, the snorkel 22 preferably extends outwardly approximately 3.5 inches and is substantially tangential to the top surface 38 of the support base 12.

The snorkel 22 also preferably includes at least one air inlet 40, air outlet 50, and passageway 52 fluidly connecting the inlets and outlets 40, 50. The terms “inlet” and “outlet” are intended to denote the flow of air through the snorkel 22 during inhalation; those skilled in the art will readily recognize that during exhalation the airflow will change. The snorkel 22 may additionally include separate passageways 52 for inhalation and exhalation, each having dedicated air inlets 40 and air outlets 50 based on the flow of air.

The air let 40 may encompass any of the various known designs of air inlets 40 and the present invention is not limited to any one particular design of an air inlet 40 unless specially claimed as such. For illustrative purposes only, the air inlet may simply feature an aperture 44 disposed proximate the distal end 46 on the top region 48 of the snorkel 22. Alternatively, the air inlet 40 may be disposed on the side and/or the back of the snorkel 22. The air inlet 40 may further include any of the known splash protectors or other devices to prevent/reduce water from being drawn into the snorkel 22.

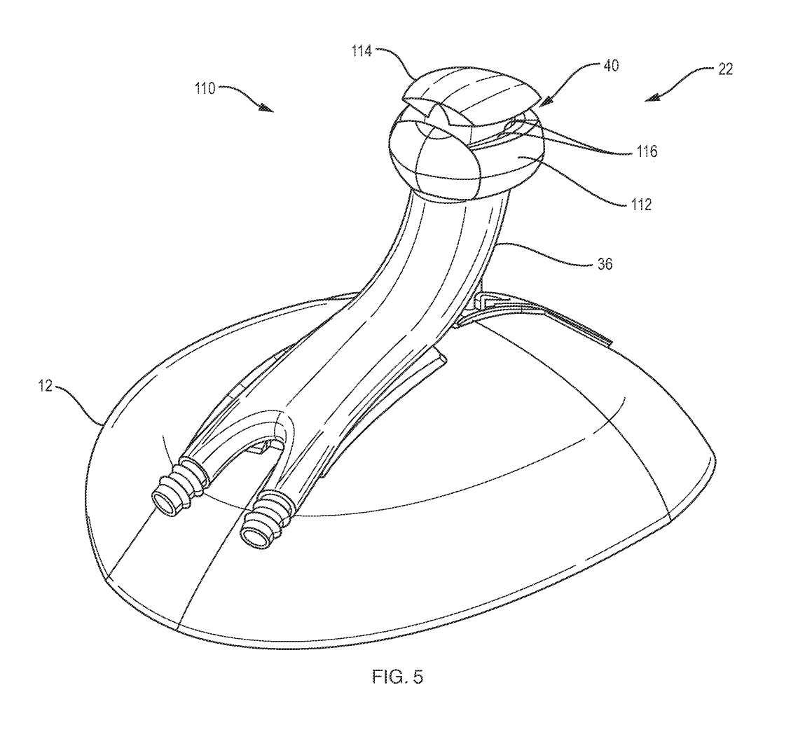

For example, the snorkel 22, FIG. 5, may feature a novel splashguard 110 that reduces the possibility of water entering the air inlet 40. The splashguard 110 preferably includes a buoyant float 112 that moves along a region of the elongated portion of the snorkel 22 and a flanged region 114. The buoyant float 112 preferably features a inversed flange region having a perimeter that substantially corresponds to at least a portion of the flanged region 114. While the air inlet is above water, the gravity causes the float 112 to move downward away from the flanged region 114, thereby opening the air inlet 40 to the atmosphere. When submerged, the float 112 moves upward against the flanged region 114, thereby sealing the air inlet 40 along at least the perimeter of the buoyant float 112 and preventing water from entering the passageway 52. The float 112 and/or the flanged region 114 may include a seal 116 such as, but not limited to, an o-ring type seal to further prevent water from entering the passageway 52.

In the preferred embodiment, the air outlet 50 are disposed proximate the support base 12, preferably proximate a back or lower region 51 of the support base 12 such that when the improved aquatic device 10 is worn on the user's head 16, the air outlets 50 are generally facing towards the neck 25 of the user's head 16. One or more flexible tubes 52, FIGS. 1 and 4, are connected (preferably removably connected) to the air outlets 50 and to the mouthpiece 54 (FIG. 1). The flexible tubes 52 may also feature inline connectors disposed a distance away from either the mouthpiece 54 or the air outlets 50 to facilitate removal or installation of the improved aquatic device 10 on the user. As best shown in FIG. 1, the flexible tubes 52 are preferably of a sufficient length such when the mouth piece 54 is disposed in the user's mouth, the flexible tubes 52 go behind the user's head 16, around the user's chin, and connect with the mouth piece 54. Having the flexible tube 52 disposed in this manner increases the overall flexibility and comfort of the improved aquatic device 10 since nothing is disposed against the user's ear 27 and significantly reduces the likelihood of the snorkel being in the way during use or getting caught on something.

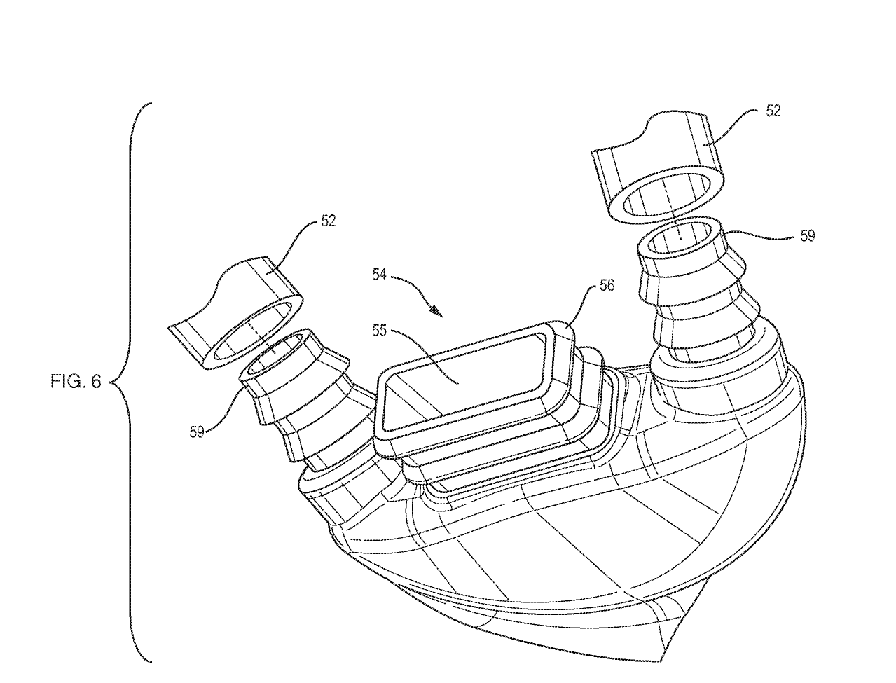

In the exemplary embodiment, the improved aquatic device 10 features a first and a second flexible tube 52 wherein each of the flexible tubes 52 is disposed on opposite sides of the user's head 16 (i.e., on the left and right side of the user's head 16) and connects to the mouthpiece 54. Referring specifically to FIG. 6, the mouthpiece 54 features a flanged region 56 having an aperture 55 sized and shaped to fit within the user's mouth as is well known. The mouthpiece 54 also preferably features at least one passageway fluidly connecting the aperture 55 of the flanged region 56 to at least one opening 59 which is sized and shaped to be secured to the flexible tubes 52, thereby forming an overall passageway between the aperture 55 in the mouthpiece 54 and the air inlet 40 of the snorkel 22. The opening 59 preferably features a flanged region and/or a connector such that the flexible tubes 52 can be removably secured/connected to the mouthpiece 54. Alternatively, the flexible tube 52 may be permanently secured to the mouthpiece 54. The mouthpiece 54 also preferably includes a purge valve (not shown) as is well known to those skilled in the art.

This embodiment further increases the comfort in numerous ways. For example, the user of two flexible hoses 52 equalizes the drag on both sides of the user's head 16 and also reduces the overall drag since the flexible hoses 52 are generally out of the flow of the water since most of the flexible tubes are beneath the user's head 16 unlike the known snorkels which along the side of the user's head and therefore directly in the flow of the water. Additionally, having two flexible tubes 52 allows the mouthpiece to hang close to the user's mouth when not in use (in contrast to the known snorkel designs in which the mouthpiece dangles about one side of the user's head 16) and allows the diameters of each flexible tube to be reduced while still maintaining a comparable overall airflow rate and pressure drop. Moreover, the use of two separate flexible tubes is particularly suited to applications having separate inhalation and exhalation passageways as described above.

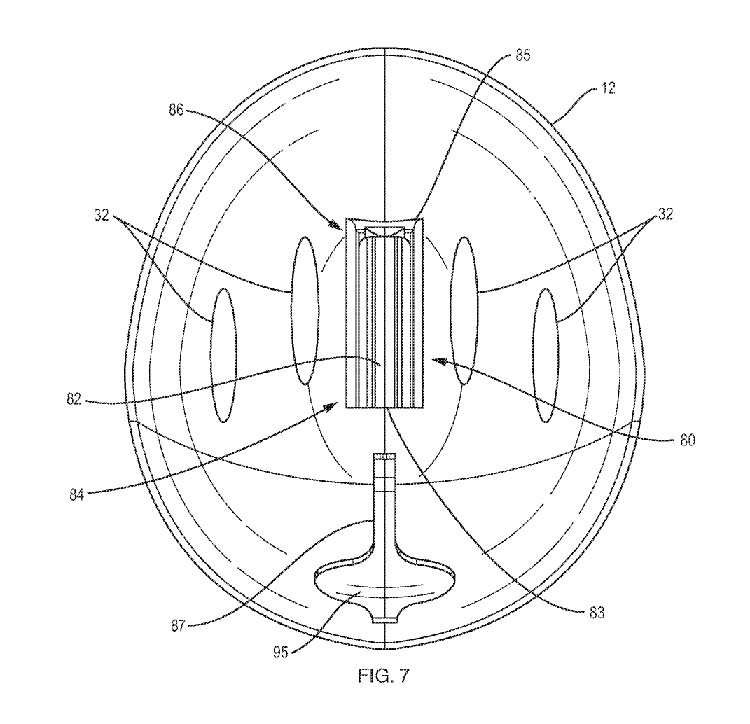

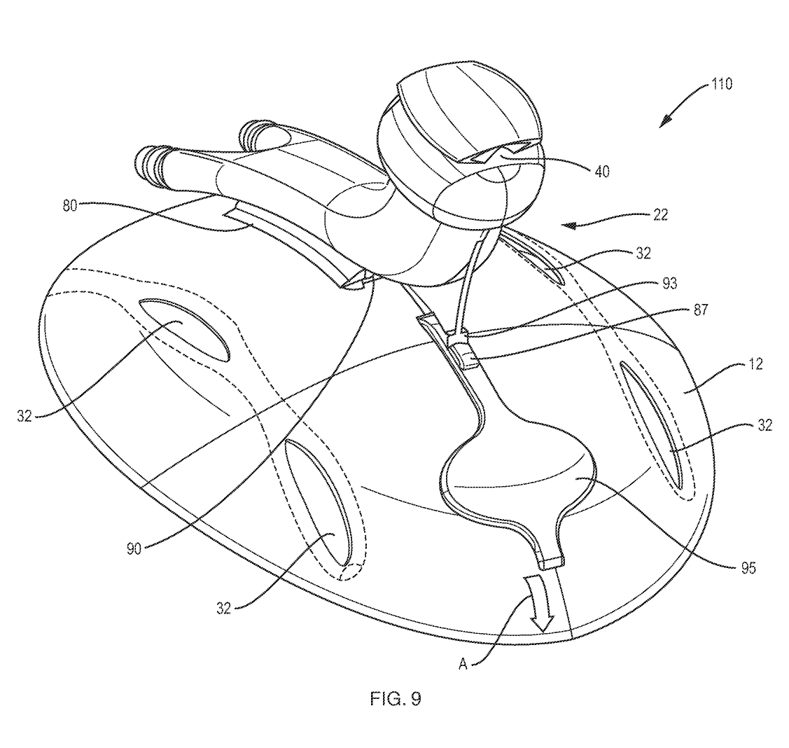

According to a preferred embodiment, the support base 12, FIGS. 7 and 8, of the improved aquatic device 10 optionally features at least one connection system 80 that allows various equipment to be secured to the support base 12 as will be explained in greater detail hereinbelow. For illustrative purposes only, the connection system will be explained in conjunction with a snorkel 22 (FIG. 9), though this is not intended to be a limited of the present invention unless otherwise specifically claimed as such. It is important to note that the improved aquatic device 10 may feature multiple connection systems 80 disposed about the support base 12 such that two or more pieces of equipment (such as, but not limited to, a snorkel 22 and/or a light 100) may simultaneously be connected to the support base 12.

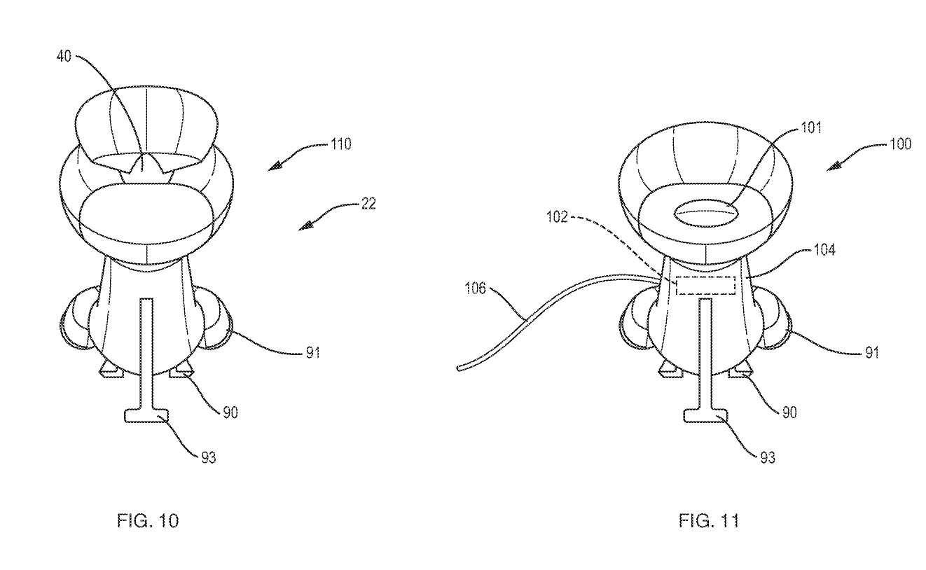

The connection system 80 may feature any device known to those skilled in the art such as, but not limited to, various fasteners including a hook and loop type fasteners, magnetic fasteners, suction fasteners, clamp fasteners, and other known mechanical fasteners. In the preferred embodiment, the connection system 80 features at least one channel, groove, or slot 82 (best seen in FIG. 8) such as, but not limited to, a dovetail type slot, having an opening 83 disposed proximate a first end 84 of the channel 82 and a flanged or stopper 85 disposed proximate a second end 86 of the channel 82. The various equipment (such as the snorkel 22, FIG. 10), preferably feature a protrusion or pin 90, FIGS. 9 and 10) disposed proximate the base 91 of the snorkel 22 sized and shaped to fit within the groove 82 of the connection system 80 in the support base 12. Optionally, a biased tab 87 (FIGS. 7 and 9) may be used to secure/lock the various equipment within the channel 82 by applying a force against the base 91 of the snorkel 22 urging the snorkel 22 against the flanged region 85 of the channel 82. The base 91 of the snorkel 22 may feature a protrusion 93 disposed proximate the first end 82 of the channel 82 that the bias tab 87 applies the force against. The bias tab 87 may optionally feature an enlarged region 95 to facilitate moving the biased tab 87 in the direction of arrow A in order to release the biased tab 87. This enlarged region 95 is particularly advantageous since divers often wear thick gloves to protect their hands.

According to another embodiment, the improved aquatic device 10 may feature a light 100, FIG. 11, that may be secured to the support base 12 using any of the connection systems 80 described above. Alternatively, the light 100 may be permanently secured to the support base 12 such that the light 100 is an integral piece of the support base 12. The light 100 preferably includes a light source 101 (such as, but not limited to, an LED or traditional bulb) and a power source 102 (such as a battery or the like). The power source 102 may be disposed within the body 104 of the light 100. Alternatively, the power source 102 may be disposed remotely and connected to the light 100 by one or more wires 106. For illustrative purposes only, the power source 102 may be secured to the dive tanks, buoyancy-compensators device (BV), and/or other gauges.

The use of a light 100 secured to the support base 12 of the present invention eliminates the requirement of the user having to hold a light in their hand, thereby freeing one of the user's hands and eliminating the possibility of the user accidentally dropping the light. Moreover, because the light 100 is secured to the user's head 16, the light 100 will move with the user's head 16 ensuring that the light 100 is always pointing in the direction that the user is looking.

The light 100 may also feature a light (such as a 360 degree light) used primarily for identification purposes. As discussed above, identifying an individual while in the water is often difficult because of the reduced visibility and ability to communication as well as common, non-descript wetsuits. The light 100 may feature multiple light sources 101 in various colors and which blink in various patterns. The use of various colors and blinking patterns allows users to quickly identify a specific individual from others, and also facilitates finding the individual while under water. While the snorkel 22 and the light 100 have been described separately, this is not a limitation of the present invention and the two may be combined.

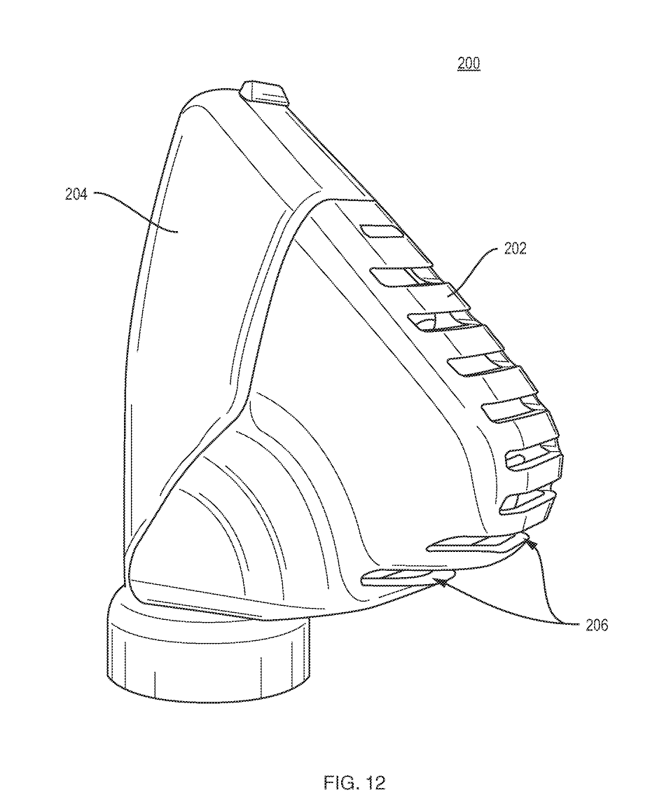

Another feature of the present invention is a dry top 200, FIG. 12 that serves to prevent water from entering the snorkel when the user is under water, irrespective of the position of the user under water. The dry top 200 includes a float mechanism cover 202 which is attached to the remainder of the body 204 of the dry top by means of friction fit, adhesive or the like. The cover 202 includes a number of slots or openings 206 into and out of which flows water.

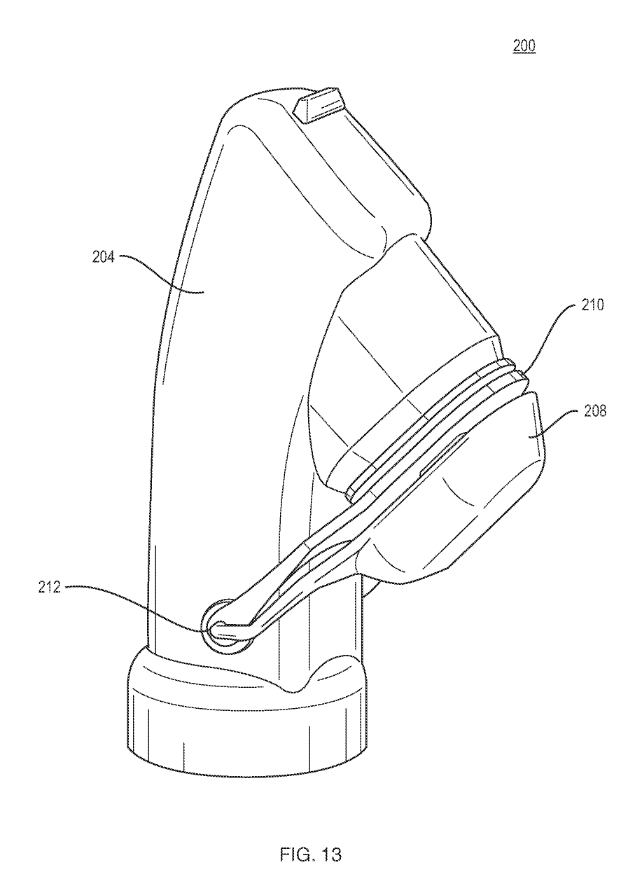

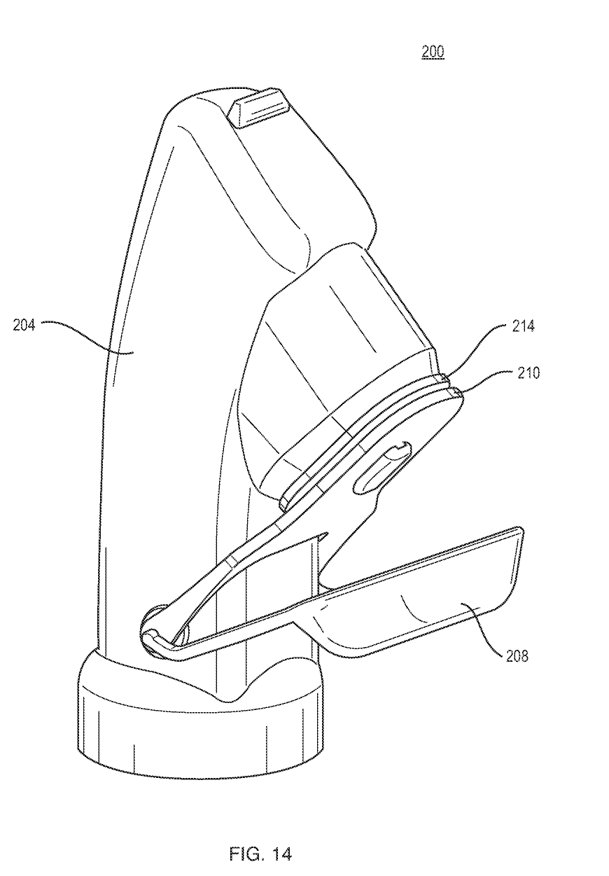

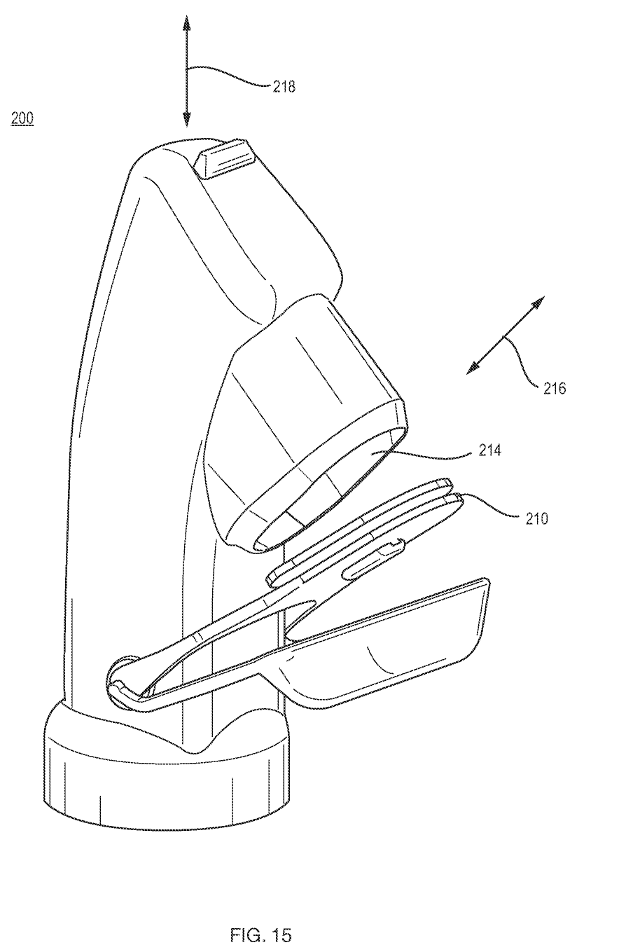

As shown in greater detail in FIG. 13 with the cover 202 removed, the dry top 200 includes a float mechanism 208 and an inlet closure mechanism 210. The float mechanism 208 and the inlet closure mechanism 210 operate independently but are coupled to the dry top body 204 at the same pivot axis 212. As shown in FIG. 14, when in use and dependent on the users position under water, the float mechanism 208 may fall back away from the closure mechanism 210 however the water pressure will always keep closure mechanism 210 closed and seated against the inlet opening 214 irrespective of the position of the user (i.e. whether the user is upside down or on his or her side).

In a preferred embodiment, the dry top body 204 is made of a generally hard plastic material while the float mechanism 208 and the closure mechanism 210 may be made of a softer, more resilient material which allows the float mechanism to remain buoyant and the closure mechanism 210 to effectively seal the opening 214 of the dry top 204.

As shown in FIG. 15, once the dry top is out of the water, the closure mechanism 210 falls away from inlet opening 214 pulling down on the closure mechanism 210 and the fact that the dry top opening 214 is oriented along an axis 216 that is disposed at approximately a 45° angle to the longitudinal axis 218 of the remainder of the dry top 200.

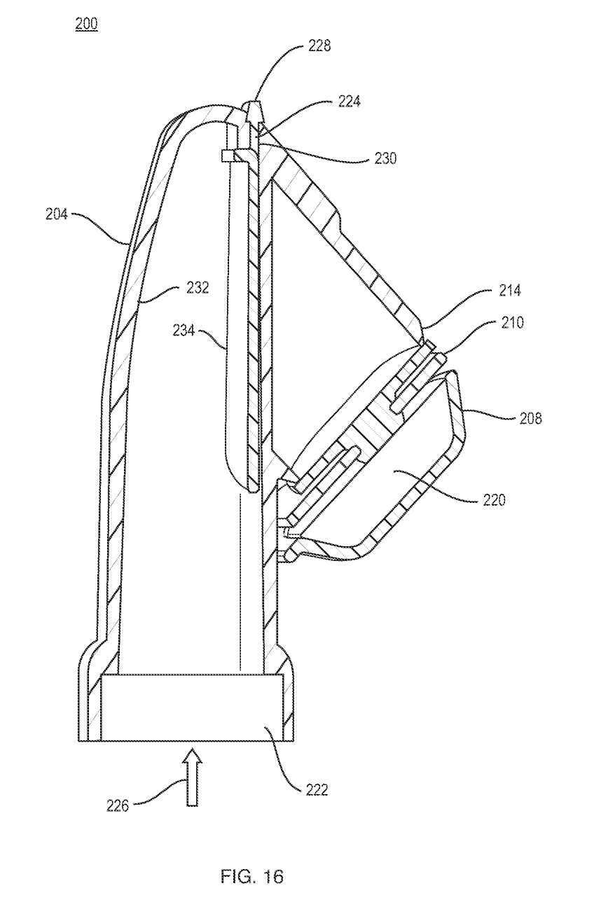

The dry top 200, FIG. 16, as shown in cross section illustrating how the hollow interior region 220 of the float mechanism 208 causes the float mechanism to act as a float, while the closure mechanism 210 includes one or more layers which serve to effectively provide a water tight seal against the opening 214 of the dry top, which is connected proximate end 222 to a snorkel or other type of breathing tube.

An additional, optional feature of the dry top 200 according to the present invention is the provision of a unidirectional, rubber closure flap 224. In the embodiment of an aquatic device wherein a separate air inlet and air exhaust path is provided, closure flap 224 ensures that airway passage 226 only provides for fresh, inlet air. In operation, closure flap 224 includes a small tab region 228 that is inserted into a small slot or passageway 230 at the top of the dry top 200. Closure flap 224 is an optional, flexible, rubberlike material. When a user takes in a breath of fresh air (assuming the closure mechanism 210 is not in position), the closure flap 224 is forced open in the direction of arrow 232 allowing air to enter the dry top and the snorkel device to which it is attached. When the user exhales, air pressure in the direction of arrow 234 closes closure flap 224 and prevents air from escaping the dry top. In this embodiment, a separate air exhaust path is provided as will be explained in greater detail below. Using such a mechanism helps insure that the air intake passageways of a snorkel device will contain only fresh air and not containing recycled air expelled from the users lungs which carry much less oxygen and therefore provides much less benefit to the user. Although shown in FIG. 16, the air inlet closure flap 224 is optional and need not be provided if there is not a dual path air inlet/air exhaust mechanism. The dry top body 204 may not include opening 230 in that embodiment or alternatively, a small rubber plug may be inserted into that opening.

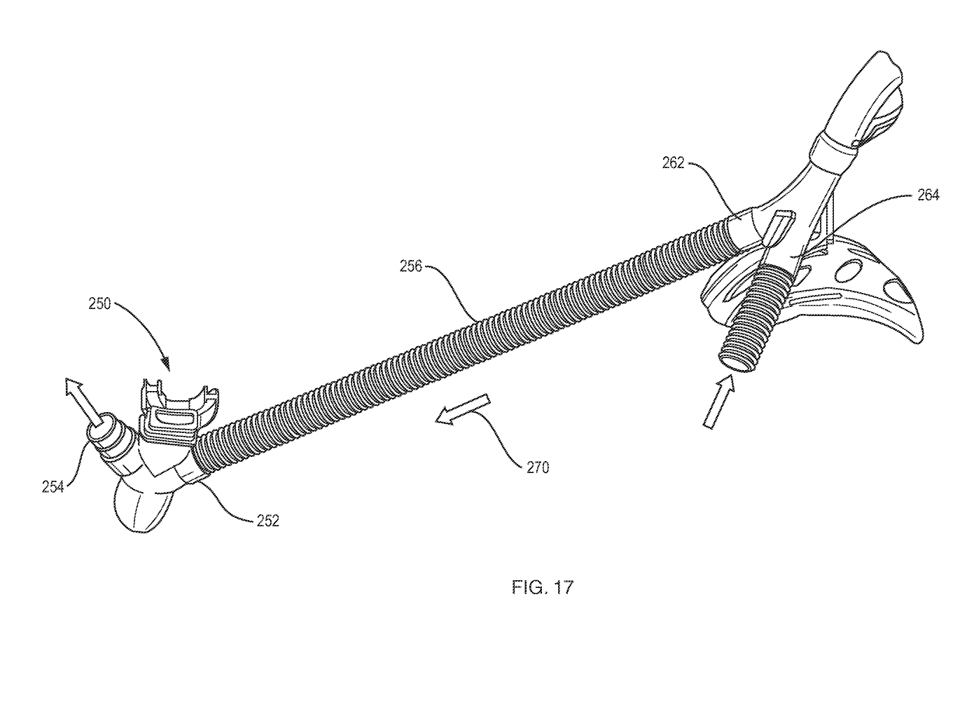

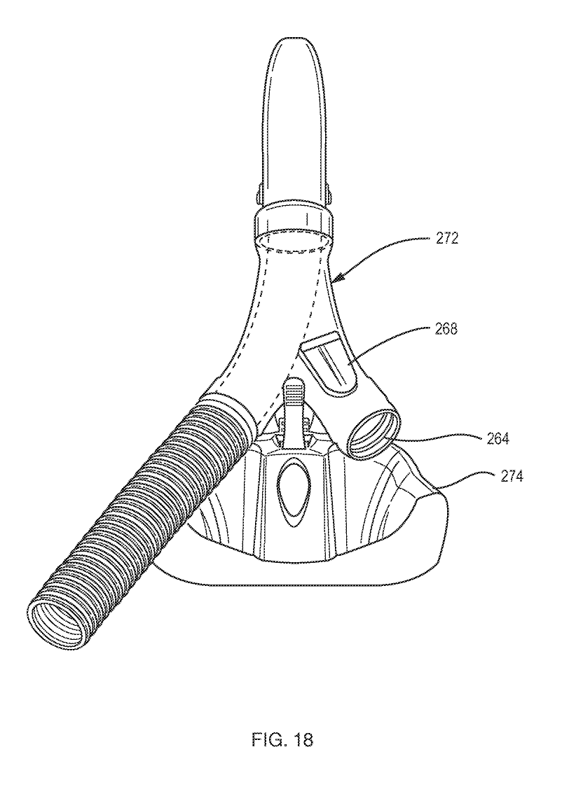

As mentioned above, one feature of the present invention is the inclusion of a separate air inlet and air exhaust path to insure that a user does not re-breath previously used air. In such an embodiment, the mouthpiece 250, FIG. 17 that a user inserts into his or her mouth includes an air inlet port 252 and an air exhaust port 254. The air inlet port 252 is coupled to the air inlet region 262 of the other portion of the aquatic device by means of a flexible tube 256 as is well known in the art. Although not shown, a similar flexible tube connects the air exhaust port 254 of the mouthpiece 252 to the air exhaust port 264 of the headgear portion of the aquatic device. The air exhaust port 264, FIG. 18, includes a unidirectional closure of valve 268.

In use, remembering that the dry top includes an air inlet closure valve preventing exhaust the air from escaping through the dry top, the configuration of the present invention shown in FIG. 17 allows the user to breathing in fresh air from the inlet portion of the system as shown generally by arrow 270 which represents air flow through the inlet tube 256. When a user exhales, the closure valve in the dry top prevents air from escaping through the inlet tube 256 while allowing the exhaust air from mouthpiece 252 easily flow from exhaust port 254 in the mouthpiece through a connecting tube, to the exhaust port 264 of the headpiece 272 mounted on to the head plate 274. Valve 268 opens allowing exhaust air to escape from the aquatic device thus maintaining a separate air inlet and air exhaust path. Although less desirable, the two or dual air port dry top may be configured as one port with both air passages connected together.

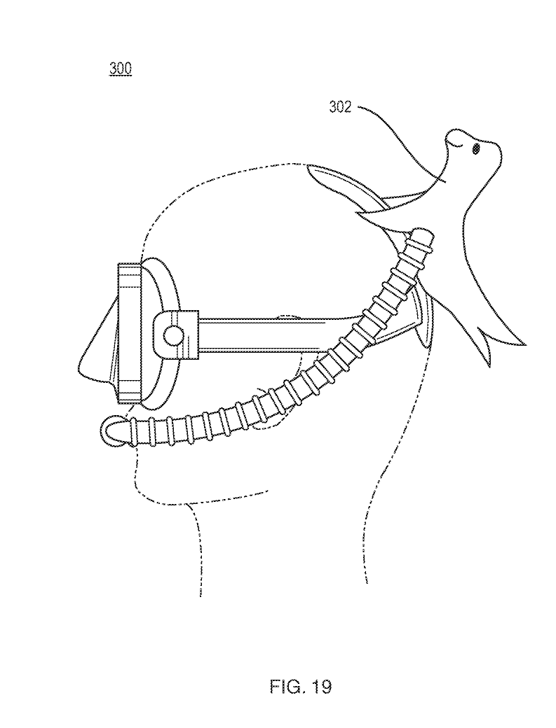

In yet another embodiment, the aquatic headgear 300, FIG. 19, according to the present invention may include a figure, character or animal 302 which may be an integral part of the snorkel as shown, or the dry top elements may be designed into the figure itself. Alternatively, the FIG. 302 may be a separate element holding, straddling or sitting on the snorkel. A dry top as described herein may also be incorporated into the FIG. 302.

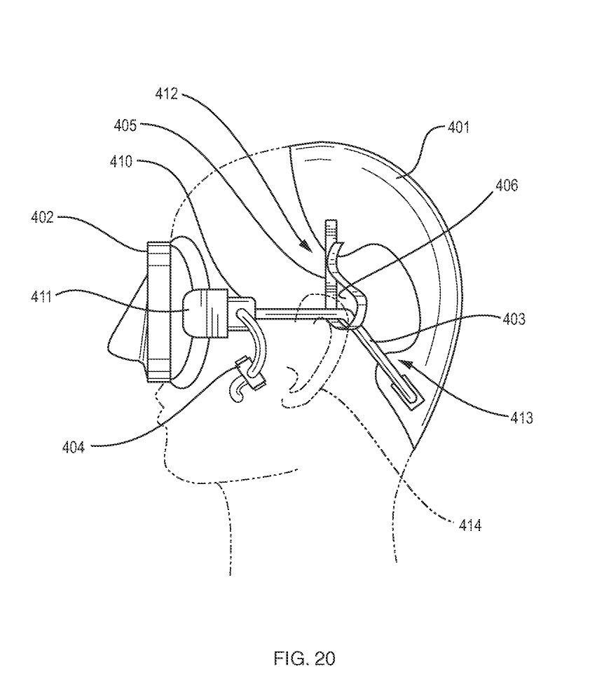

The support base of the present invention is preferably a neoprene or rubber headpiece section 401, FIG. 20, designed to extend from approximately the middle of the top of the head, to the suboccipital region. Its shape is roughly that of the posterior section of a divers hood and is designed to fit comfortably around the posterior and superior section of a person's head. It also preferably is elastic enough to conform to most head shapes, and provide the needed elasticity for eventually securing a mask or goggles 402 to the front of the users face. The headpiece 401 is shaped to extend around the sides of the head substantially at the superior mid section 412 and the occipital region 413 so as to provide a secure fit on the head of the user.

Two straps or cords (preferably nylon) 403 are attached on either side of the inferior (suboccipital) region of the headpiece. The opposite ends of these straps will be adjustably connected to the sides of the mask or goggles by any well-known means, but preferably with cord stops 404 such as those used to cinch up hood straps on winter parkas.

Two more straps, cords, or the like 405 are securely attached to the lateral and superior aspects of the headpiece 401. At their distal ends, the straps will form a fixed, or adjustable (up or down) channel 406 for the two mask straps (one on each side attached to the occipital portion) to pass through.

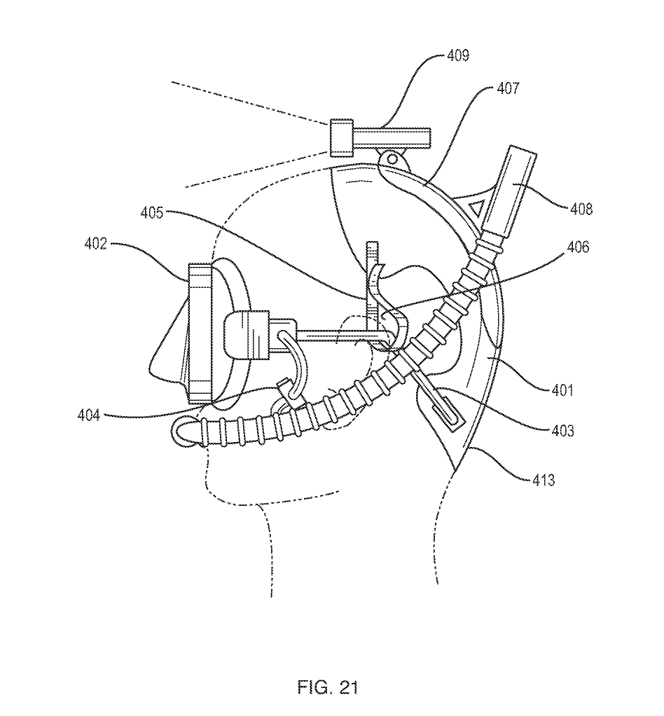

The headpiece section 401 will allow for a secure, contoured and comfortable base on which a mounting system 407, FIG. 21 will be either permanently or detachably secured for the purpose of removably attaching objects such as snorkels 408, cameras, lights 409, etc.

In its assembled and ready to use state, the two mask straps 403 (preferably round nylon cords), that are attached at one end to the occipital portion 413 of the headpiece 401 are directed to pass through channels 406 formed by the straps attached to two sides of the superior and lateral aspect of the headpiece 401.

The two mask cords or straps 403 are then directed, one on each side of the mask, where they either pass through either the pre-existing strap adjustment mechanism, or preferably through a hole in a separate adapter strap 410 that is fed through the strap adjuster 411 and brought back on itself.

The cords or straps 403 are preferably directed though the mask adjusters 410 so that their free ends are facing outwards. The free ends of the cords preferably then pass through a simple cord stop 404 allowing for easy adjustment of the cord length.

To use the device, the user places the headpiece 401 on the head so that it covers the superior head to approximately the middle, and the posterior surface to the suboccipital region. The user then places his/her mask 402 optimally on the face and tightens the mask cords 403 until the mask is comfortably secured on the face. If needed, the two straps at the superior lateral aspect of the headpiece 405 can be shortened so as to raise the channels that the mask cord 403 passes through. This will both direct the mask cord 403 so that is passes comfortably behind the users ears, and apply slightly more downward pressure on the headpiece section covering the top of the head so as to secure it more firmly to the user's head.

Once the user has successfully adjusted the strap mechanism, all she/he needs to do for subsequent placement and removal of the system is adjust the mask cords 403 to the desired comfort.

When tightening the mask cord 403 at the mask end, the neoprene or rubber at the opposite end 401 is stretched providing a comfortable, elastic fit. Also, in this configuration, the mask cord 403 is directed above and behind the user's ears 414 for comfort and correct strap positioning and alignment on the mask 402. In addition, as the mask cord 403 is tightened, the headpiece 401 is simultaneously tightened around the superior and suboccipital regions of the head. Alternately, the mask cords 403 can be somewhat permanently affixed to the sides of the mask, and adjustably connected to the occipital portions of the headpiece (not pictured).

Although the system can be used as a mask strap only, in its preferred embodiment a mounting system 407, FIG. 21, would be attached or integrally molded to the posterior and superior surface of the headpiece 401 for mounting lights, cameras, snorkels, etc. The mounting system can be made of rubber, plastic, hook and loop fasteners, or the like, sufficient to suit its intended purpose, and can be permanently or detachably connected to the headpiece. In its preferred embodiment the mounting system 407 would be sized and shaped to fit over the posterior and superior portion of the headpiece, and shaped to accommodate the curvature of the head. If needed, ribbing or supports can be molded into the headpiece 401 at necessary locations to help support the shape and strength of the headpiece 401 and mounting base 407. If necessary, a rubber or plastic support extending from the inferior surface of the mounting system 407 to the inferior occipital aspect of the headpiece 401 may be used to help support the occipital portion of the headpiece 401 and keep it in the optimal position on the users head.

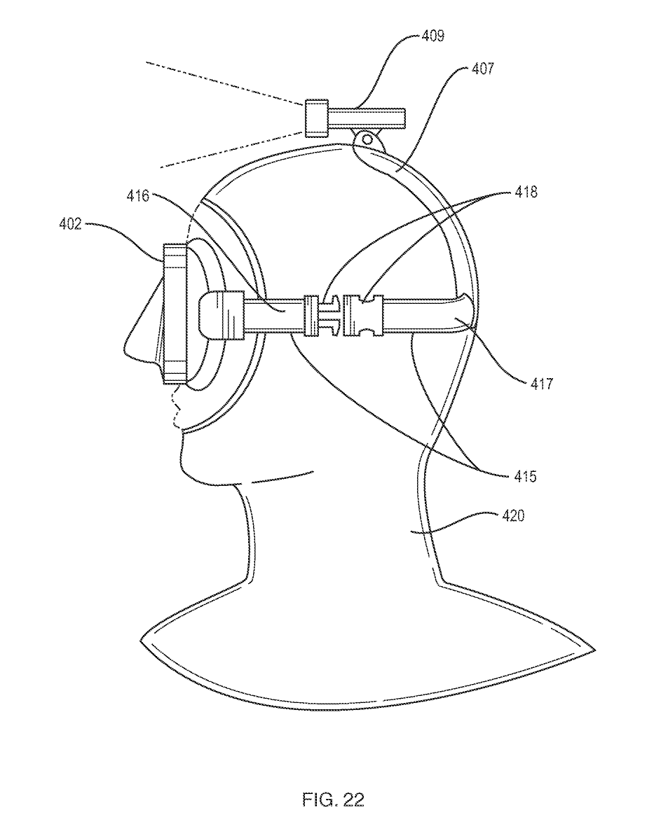

In another embodiment for a diver's hood, the mounting system 407, FIG. 22, may be permanently or removably attached to the back of a divers hood 420 for use anytime when needed. In this configuration the diver may use his or her own conventional mask straps, used normally by stretching them over his head and around the back side of his head and mounting system assembly (not shown), or alternately, separate dive straps 415 may be used. In this configuration, the strap is separated into to halves per side. An anterior half 416 and a posterior half 417 being removably connected by a quick release mechanism on both sides of the head 418. In this configuration, the posterior half of the strap 417 is permanently connected to the hood or mounting system, and the anterior half of the strap 416 is connected to the mask 402. A means for adjusting the tension on the mask straps is can be on either the anterior or posterior strap at a determined location depending on connecting or adjusting apparatus used. The two halves 416 and 417 are connected to each other via a snap or buckle system 418.

To use the system, the diver simply places the hood over his head, and then attaches the anterior straps 416 connected to his mask 402 to the posterior straps 417 already attached to the hood 420. If the user needs to take the mask off quickly, he can detach one or both straps quickly and easily.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

The invention claimed is:

1. An aquatic headgear device for use with underwater goggles, comprising:
a support base, sized and shaped to generally fit over only a posterior superior portion of a user's head;
said support base including an engagement device, for removably securing an object to said support base, said support base further including a snorkel engagement element, for removably securing a snorkel to said support base;
first and second connection straps, coupled to said support base and configured for coupling to an underwater goggle device, for connecting said supporL base to first and second goggle attachment regions on said underwater goggle device, each goggle attachment region disposed on opposite sides of said underwater goggle device;
a snorkel, removably secured to said snorkel engagement element disposed on said support base, said snorkel extending generally outwardly and away from said support base, said snorkel including an air intake aperture proximate a first end, and first and second user air passages proximate a second end, wherein said air intake aperture is fluidly coupled to a first one of said first and second user air passages, wherein said air intake aperture includes a one-way valve disposed in an interior region of said snorkel and configured for allowing the flow of air into said air intake aperture from outside said snorkel but preventing the flow of air from said first user air passage through said air intake aperture to outside said snorkel;
first and second flexible tubes, each of said first and second flexible tubes having a first end connected to one of said first and second user air passages of said snorkel respectively, and a second end connected to a user mouthpiece, and wherein said second user air passage of said snorkel includes a one-way valve configured for allowing user air exhausted through said mouthpiece and said one of said first and second flexible tubes coupled said second air passage of said snorkel to exit said snorkel through said one-way valve in said second user air passage, whereby said first one of said first and second flexible tubes serves as an air intake for a user using said snorkel and said second one of said first and second flexible tubes serves as an air exhaust tube for a user using said snorkel;
wherein said snorkel further includes a splashguard, wherein said splashguard includes:
a flanged open region disposed proximate a distal of said air intake aperture; and
a buoyant float moveably disposed about an exterior portion of said snorkel, said buoyant float configured such that when said buoyant float is above water, gravity urges said buoyant float towards a first position wherein said air intake aperture is substantially unobstructed by said buoyant float, and when said buoyant float is below water, buoyancy of said buoyant float urges said buoyant float towards a second position wherein said buoyant float engages said flanged open region and said air passageway is substantially sealed.

2. The aquatic headgear device as claimed in claim 1 wherein said support base is generally flexible.

3. The aquatic device as claimed in claim 2 wherein said support base includes a substantially concaved shape and a layer of a non-slip material disposed about an interior region of said concave shaped support base.

4. The aquatic device as claimed in claim 3 wherein said non-slip material includes neoprene.

5. The aquatic device as claimed in claim 1 wherein said object removably secured to said support base is selected from the group consisting of a light and a camera.

6. The aquatic device as claimed in claim 5 further including a power source secured to said support base and electrically connected to said light.

7. The aquatic device as claimed in claim 1, wherein said snorkel includes a generally longitudinal axis, and wherein said air intake aperture is disposed at an angle relative to said generally longitudinal axis of said snorkel.

8. The aquatic device as claimed in claim 7, wherein said angle is approximately 45°.

9. The aquatic device as claimed in claim 8, wherein said splashguard further includes a pivotable closure mechanism disposed between said flanged open region of said snorkel and said movable buoyant float, wherein said pivotable closure mechanism and said movable buoyant float are both configured for pivoting about an axis that is transverse to the generally longitudinal axis of said snorkel, said pivotable closure mechanism and movable buoyant float configured such that when said movable buoyant float is above water, gravity urges said buoyant float towards a first position wherein said air intake aperture is substantially unobstructed by said closure mechanism and said buoyant float, and when said buoyant float is below water, buoyancy of said buoyant float urges said buoyant float towards a second position wherein said buoyant float urges said closure mechanism into an engaged position with said flanged open region of said snorkel, whereby said air passageway of said snorkel is substantially sealed.

10. The aquatic device as claimed in claim 9, wherein said pivotable closure mechanism and said movable buoyant float are both configured for pivoting about the same axis.

11. The aquatic device as claimed in claim in 1, wherein said support base is generally permanently affixed to an exterior region of a diving hood.

12. An aquatic headgear device for use with underwater goggles, comprising:
a support base, sized and shaped to generally fit over only a posterior superior portion of a user's head;
said support base including an engagement device, for removably securing an object to said support base, said support base further including a snorkel engagement element, for removably securing a snorkel to said support base;
first and second connection straps, coupled to said support base and configured for coupling to an underwater goggle device, for connecting said support base to first and second goggle attachment regions on said underwater goggle device, each goggle attachment region disposed on opposite sides of said underwater goggle device;
a snorkel, removably secured to said snorkel engagement element disposed on said support base, said snorkel extending generally outwardly and away from said support base, said snorkel including an air intake aperture proximate a first end, and first and second user air passages proximate a second end, wherein said snorkel includes a generally longitudinal axis, and wherein said air intake aperture is disposed at an angle relative to said generally longitudinal axis of said snorkel, wherein said air intake aperture is fluidly coupled to a first one of said first and second user air passages, wherein said air intake aperture includes a one-way valve disposed in an interior region of said snorkel and configured for allowing the flow of air into said air intake aperture from outside said snorkel but preventing the flow of air from said first user air passage through said air intake aperture to outside said snorkel;
first and second flexible tubes, each of said first and second flexible tubes having a first end connected to one of said first and second user air passages of said snorkel respectively, and a second end connected to a user mouthpiece, and wherein said second user air passage of said snorkel includes a one-way valve configured for allowing user air exhausted through said mouthpiece and said one of said first and second flexible tubes coupled said second air passage of said snorkel to exit said snorkel through said one-way valve in said second user air passage, whereby said first one of said first and second flexible tubes serves as an air intake for a user using said snorkel and said second one of said first and second flexible tubes serves as an air exhaust tube for a user using said snorkel;
wherein said snorkel further includes a splashguard, wherein said splashguard includes:
a flanged open region disposed proximate a distal of said air intake aperture; and
a pivotable closure mechanism disposed between said flanged open region of said snorkel and a movable buoyant float, wherein said pivotable closure mechanism and said movable buoyant float are both configured for pivoting about an axis that is transverse to the generally longitudinal axis of said snorkel, said pivotable closure mechanism and movable buoyant float configured such that when said movable buoyant float is above water, gravity urges said buoyant float towards a first position wherein said air intake aperture is substantially unobstructed by said closure mechanism and said buoyant float, and when said buoyant float is below water, buoyancy of said buoyant float urges said buoyant float towards a second position wherein said buoyant float urges said closure mechanism into an engaged position with said flanged open region of said snorkel, whereby said air passageway of said snorkel is substantially sealed.

13. The aquatic headgear device of claim 12, wherein said angle is approximately 45°.

14. The aquatic headgear device of claim 12, wherein said support base is generally permanently attached to a diving hood.
